(19) United States
(12) Patent Application Publication
(10) Pub. No.: US 2025/0248404 A1
(43) Pub. Date: Aug. 7, 2025

(54) BACTERIA-BASED AGRICULTURAL BIOFERTILIZER COMPOSITION AND THE METHOD TO IMPROVE SOIL HEALTH

(71) Applicants: BENOY BERRY, LONDON (GB); PRAVEEN KUMAR DIVVELA, WEST GODAVARI (IN)

(72) Inventors: BENOY BERRY, LONDON (GB); PRAVEEN KUMAR DIVVELA, WEST GODAVARI (IN)

(21) Appl. No.: 19/091,928

(22) Filed: Mar. 27, 2025

(30) Foreign Application Priority Data
Mar. 30, 2024 (GB) GB2404616.1

(51) Int. Cl. A01N 63/22; A01N 25/02; A01N 25/10; A01P 21/00; C12N 1/20; C12R 1/11

(52) U.S. Cl. CPC A01N 63/22; A01N 25/02; A01N 25/10; A01P 21/00; C12N 1/205; C12R 2001/11

(57) ABSTRACT

Exemplary embodiments of the present disclosure are directed towards a carrier-based agricultural biofertilizer composition, comprising an isolated Bacillus megaterium strain CGAPGPBBS-034 having the deposit accession number KY495205; and an agriculturally acceptable liquid formulation with a stabilizer to provide stability and support for at least one of the Bacillus species, thereby enhancing the growth and overall health of the plants.

### CROSS-REFERENCE TO RELATED APPLICATIONS

This patent application claims priority benefit of UK Patent Application No: GB2404616.1, entitled “A BACTERIA-BASED AGRICULTURAL BIOFERTILIZER COMPOSITION AND THE METHOD TO IMPROVE SOIL HEALTH”, filed on 30 Mar. 2024. The entire contents of the patent application are hereby incorporated by reference herein in its entirety.

### TECHNICAL FIELD

The present disclosure generally relates to microbial biofertilizer compositions, more particularly the invention relates to a carrier-based agricultural biofertilizer composition and a method for enhancing soil using the biofertilizer composition.

### BACKGROUND

Agricultural biofertilizers can benefit from ongoing developmental research to further enhance their effectiveness and contribute to sustainable agriculture. Here are some key areas of research that can support the development and optimization of agricultural biofertilizers:
a) Microbial diversity: Research can focus on understanding the diversity and functionality of beneficial microorganisms present in biofertilizers. By identifying specific strains or consortia of microorganisms that promote plant growth, nutrient uptake, and soil health, researchers can develop biofertilizers with improved performance.
b) Formulation and application methods: Studying the formulation and application methods of biofertilizers can help optimize their effectiveness. Research can explore different carriers or substrates to enhance microbial survival and activity during storage and application. Additionally, studies can investigate the optimal application rates, timings, and techniques for different crops and soil types.
c) Compatibility with other inputs: Research can assess the compatibility of biofertilizers with other agricultural inputs, such as synthetic fertilizers, pesticides, and biostimulants. Understanding the interactions between biofertilizers and these inputs can help farmers optimize their use and reduce potential negative interactions.
d) Long-term effects on soil health: Long-term studies are needed to evaluate the impact of biofertilizers on soil health and ecosystem services. Research can investigate their effects on soil organic matter content, microbial communities, soil structure, and nutrient cycling over multiple cropping seasons. This information can guide sustainable soil management practices and provide insights into the long-term benefits of biofertilizers.
e) Field trials and validation: Conducting field trials in different agroecosystems and regions is crucial for validating the performance of biofertilizers under real-world conditions. Research can evaluate the effectiveness of biofertilizers in terms of crop yield, nutrient content, pest and disease resistance, and overall sustainability. These trials can also help identify specific crops, soil types, and environmental conditions where biofertilizers benefit most.
f) Economic viability and scaling up: Research can explore biofertilizer production and distribution's economic viability and scalability. Studies on cost-effectiveness, market demand, and business models can help promote the adoption of biofertilizers by farmers and facilitate their integration into existing agricultural systems.

Plant growth-promoting Bacillus (PGPB) refers to a group of beneficial bacteria that can enhance plant growth and improve crop health. These Bacillus species have the ability to colonize plant roots, stimulate root development, and provide various benefits to plants. PGPB can produce enzymes, such as phosphate solubilizing enzymes, that break down insoluble forms of nutrients in the soil, making them more accessible to plants. Bacillus species can also fix atmospheric nitrogen, converting it into a plant-usable form. This improves nutrient availability and uptake by crops, leading to healthier plants with improved growth and productivity. Certain strains of Bacillus have antagonistic properties against plant pathogens. They can produce antibiotics and antimicrobial compounds that inhibit the growth of harmful pathogens, including bacteria, fungi, and nematodes. By suppressing pathogen populations, PGPB help protect crops from diseases and reduce the reliance on chemical pesticides. Bacillus species can induce systemic resistance in plants, activating the plant's natural defense mechanisms against diseases. They can trigger the production of pathogenesis-related proteins, phytohormones, and other defence compounds, enhancing the plant's ability to withstand various stresses, such as pathogen attacks, drought, and salinity. PGPB can help plants tolerate and recover from abiotic stresses, such as drought, heat, and salinity. Bacillus species produce osmoprotectants and stress-related enzymes that alleviate the negative effects of these stresses on plants. They can also improve water and nutrient uptake efficiency, enabling crops to better withstand challenging environmental conditions. Plant growth-promoting Bacillus can contribute to overall soil health and fertility. They can enhance soil structure, nutrient cycling, and organic matter decomposition, leading to improved soil fertility and productivity. Healthy soils with a diverse microbial community, including PGPB, provide a favorable environment for plant growth and contribute to sustainable agriculture. Crop health management strategies incorporating plant growth-promoting Bacillus can help reduce the use of chemical inputs, promote ecological balance, and enhance the sustainability of agricultural systems.

Conventional agriculture, which relies heavily on chemical fertilizers, has several drawbacks and limitations that impact both the environment and long-term sustainability. Chemical fertilizers, when not properly managed, can contribute to environmental pollution. Excessive application or improper use of fertilizers can lead to nutrient runoff into water bodies, causing water pollution and eutrophication. This can result in the depletion of oxygen in aquatic ecosystems, harming fish and other aquatic organisms. Continuous use of chemical fertilizers without adequate soil management practices can lead to soil degradation. Chemical fertilizers primarily focus on supplying essential nutrients to crops but do not contribute to improving overall soil health. Over time, this can result in nutrient imbalances, reduced soil fertility, loss of beneficial soil microorganisms, and degradation of soil structure, leading to decreased crop productivity. Conventional agriculture often involves the use of synthetic pesticides to control pests and diseases. These pesticides can have unintended consequences, harming beneficial insects, birds, and other organisms, leading to a loss of biodiversity. The reduction in biodiversity can disrupt ecological balance, impacting natural pest control mechanisms and potentially leading to increased pest resistance. The production and synthesis of chemical fertilizers require large amounts of energy, predominantly derived from fossil fuels. The reliance on non-renewable resources for fertilizer production contributes to carbon emissions and further exacerbates climate change.

Additionally, the extraction and mining of phosphate and potassium, which are key components of chemical fertilizers, can lead to the depletion of finite mineral resources. Prolonged exposure to chemical fertilizers and pesticides can pose risks to human health. Farmers and agricultural workers who handle and apply these chemicals may face health hazards if not properly protected. Additionally, residues of these chemicals can remain on crops and contaminate food, potentially leading to health issues for consumers. Conventional agriculture, particularly in water-scarce regions, often involves excessive water usage. Chemical fertilizers can contribute to increased water demand due to their impact on crop growth and water requirements. This exacerbates water scarcity issues and can lead to the depletion of water resources, especially in areas with inadequate irrigation practices. To minimise these drawbacks and promote sustainable agriculture, alternative practices such as organic farming, integrated pest management, and the use of organic or bio-based fertilizers are being adopted. These approaches aim to minimize environmental impacts, promote soil health and biodiversity, reduce dependence on non-renewable resources, and prioritize the long-term sustainability of agricultural systems.

In the light of the aforementioned discussion, there exists a need for a certain system with novel methodologies that would overcome the above-mentioned disadvantages.

### BRIEF SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

An embodiment of the present disclosure is directed towards a carrier-based agricultural biofertilizer composition.

An embodiment of the present disclosure is directed towards the carrier-based agricultural biofertilizer composition includes an isolated Bacillus cereus CGAPGPBBS-048; and an agriculturally acceptable liquid stabilizer selected to provide stability and support for the Bacillus strain, thereby enhancing the growth and overall health of the plants

An embodiment of the present disclosure is directed towards developing the carrier-based agricultural biofertilizer composition that includes an isolated Bacillus cereus strain CGAPGPBBS-048 and an agriculturally acceptable liquid stabilizer, enabling enhanced plant growth and overall plant health.

An embodiment of the present disclosure directed towards the Bacillus cereus strain CGAPGPBBS-048 that exhibits plant growth-promoting abilities through the production of phytohormones, including indoleacetic acid, as well as the secretion of siderophores and other secondary metabolites that provide protection against pathogenic organisms.

An embodiment of the present disclosure directed towards the Bacillus cereus strain that may effectively produce the phytohormone to enhance plant growth and increase germination rates.

An embodiment of the present disclosure directed towards selecting liquid and solid formulations with a stabilizer allows the bacterial cells to stay stable in the plant rhizosphere for a long time, improving plant health.

An embodiment of the present disclosure is directed towards isolating Bacillus cereus strain CGAPGPBBS-048 from alkaline calcareous soil in the rhizosphere and rhizoplane from Yola, Nigeria, for use in the biofertilizer composition.

An embodiment of the present disclosure is directed towards isolating Bacillus cereus strain CGAPGPBBS-048 using a serial dilution technique and spread plate method, ensuring the purity and viability of the strain for effective biofertilizer formulation.

An objective of the present disclosure is directed toward enabling the capability of Bacillus cereus strain CGAPGPBBS-048 to produce phytohormone (indole acetic acid) and improve plant germination and growth.

An objective of the present disclosure is directed towards harnessing the plant growth-stimulating properties of Bacillus cereus strain CGAPGPBBS-048 by producing phytohormones, specifically indoleacetic acid, which regulates plant growth, improves root development, and enhances overall plant vigor.

An embodiment of the present disclosure is directed towards establishing a method for enhancing soil and plant health using a biofertilizer composition, involving the preparation of a nutrient medium, fermentation of Bacillus cereus strain CGAPGPBBS-048, formulation with adjuvants, and application to soil and plant to enhance its quality.

Furthermore, the objects and advantages of this invention will become apparent from the following description and the accompanying annexed drawings.

### BRIEF DESCRIPTION OF DRAWINGS

The invention is more fully appreciated in connection with the following details. Other objects and advantages of the present invention will become apparent to those skilled in the art upon reading the following detailed description of the preferred embodiments, in conjunction with the accompanying drawings, wherein like reference numerals have been used to designate like elements. However, for clear illustration, reference numeral of the same element in different figures might be omitted.

FIG. 1 is a flow diagram depicting a method for enhancing soil using a biofertilizer composition.

### DETAILED DESCRIPTION

It is to be understood that the present disclosure is not limited in its application to the details of construction and the arrangement of components outlined in the following description or illustrated in the drawings. The present disclosure is capable of other embodiments and of being practiced or of being carried out in various ways. Also, it is to be understood that the phraseology and terminology used herein is for the purpose of description and should not be regarded as limiting. While the invention has been described by way of examples and in terms of preferred embodiments, it is to be understood that the invention is not limited thereto. Hence the present invention may be applied in various modifications other than the embodiment. The main characters of the embodiment will be illustrated in clear and simple way. Besides, not all of the characters of the embodiment have shown in figures. The figures included herein are illustrated diagrammatically and not drawn to scale, as they are provided as qualitative illustration of the concept of the present invention.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and the like, herein do not denote any order, quantity, or importance, but rather are used to distinguish one element from another.

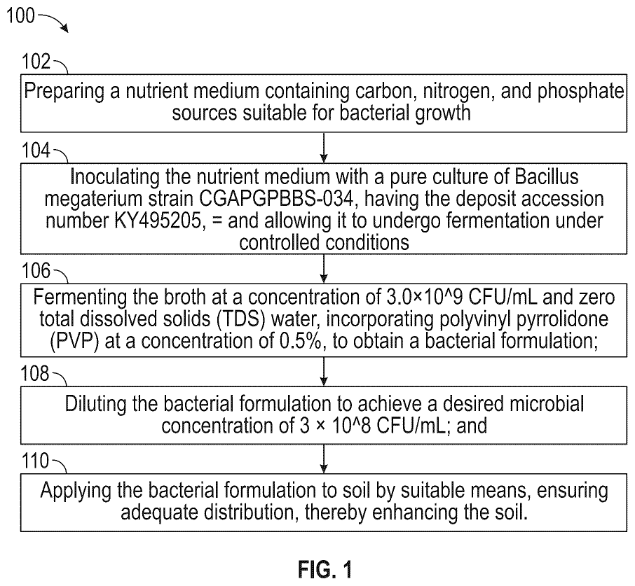

Referring to FIG. 1, 100 is a flow diagram depicting a method for enhancing soil using a biofertilizer composition. The method starts at step 102, preparing a nutrient medium containing carbon, nitrogen, and phosphate sources suitable for bacterial growth. At step 104, inoculating the nutrient medium with a pure culture of Bacillus megaterium strain CGAPGPBBS-034, having the deposit accession number KY495205, and allow it to undergo fermentation under controlled conditions. Fermenting the broth at a concentration of 3.0×10\hat{ }9 CFU/mL and zero total dissolved solids (TDS) water, incorporating polyvinyl pyrrolidone (PVP) at a concentration of 0.5%, to obtain a bacterial formulation at step 106. Diluting the bacterial formulation to achieve a desired microbial concentration of 3×10\hat{ }8 CFU/mL at step 108. At step 110, applying the bacterial formulation to soil by suitable means, ensuring adequate distribution, thereby enhancing the soil.

In an exemplary embodiment, an isolated Bacillus cereus strain CGAPGPBBS-048 may be derived from the alkaline calcareous soil found in a cowpea farming field located in Yola, Nigeria. This strain may have been incorporated into a biofertilizer formulation designed to significantly enhance seed germination in soils with higher pH levels. The chosen strain demonstrates its efficacy in enhancing plant growth and promoting overall plant health by effectively producing IAA, specifically benefiting crops such as Cowpea (Vigna unguiculata), Groundnut (Arachis hypogaea), and Soybean (Glycine max) during both rainy and dry seasons.

Morphological Characteristics of the Bacillus cereus strain CGAPGPBBS-048:
Cell shape: Spherical colonies,
Cell size: 2.5 mm
Cell arrangement: rod arrangement
Gram stain: Positive
Motility: Yes
Pigment: Absent
Capsule: Absent
Spores: Endospore formation

Physiological Properties of the Bacillus cereus strain CGAPGPBBS-048:
Behavior to oxygen: Aerobic or facultative anaerobic
Conditions for growth: pH-6.5-8.5
Temperature −37±2° C.

In another exemplary embodiment, specific strains of advantageous bacteria may not naturally occur in a given field soil or, if present, may exist in limited numbers or exhibit reduced activity, thereby failing to impart any beneficial effects on plants in an unaltered or unenhanced rhizosphere. This is true regardless of the inherent valuable traits possessed by these bacteria.

In order for a bacterial strain with inherent beneficial traits, such as a plant growth-promoting rhizobacteria (PGPR), to exert a positive impact on plant growth, it must possess a competitive advantage and be a robust colonizer within the rhizosphere during active plant growth. It should be noted that without modifications or enhancements, it may highly improbable for any native or naturally occurring PGPR, including the bacterial strain CGAPGPBBS-034, to confer benefits to plant growth. This highlights the importance of creating conditions that favor the colonization and effectiveness of PGPR strains.

In another exemplary embodiment, the PGPR strain CGAPGPBBS-034 may have been obtained from the rhizosphere and may show a beneficial trait of solubilizing phosphorus in higher pH conditions, enhancing plant growth. Furthermore, laboratory cultivation techniques may have been utilized to optimize the growth and population density of CGAPGPBBS-034, thereby maximizing its PGPR properties. These specific growth conditions may have been identified to enhance the competitive advantage of CGAPGPBBS-034 when applied to the rhizosphere, resulting in a positive impact on plant growth. In essence, the determination of growth conditions that promote the successful colonization of the rhizosphere by CGAPGPBBS-034 has enabled its beneficial effects on plant growth, which would not be achievable under normal circumstances.

In another exemplary embodiment, a biologically pure culture of CGAPGPBBS-034 may be grown to prepare a stock culture. Aliquots of this stock culture were preserved in cryogenic vials in a −80° C. For production runs, frozen stock culture may be used to inoculate a flask containing nutrient broth media and under specific conditions.

In another exemplary embodiment, the bacterial culture may be cultivated at a temperature range of 30-32° C. during the rainy season, and in alternative embodiments, at a temperature range of 48-50° C. during the dry season. The flask culture is then scaled up in a fermenter under similar growth conditions, resulting in increased population growth and enhanced plant growth-promoting properties of CGAPGPBBS-034 as the culture reaches the early stationary phase. Aliquots from the carly stationary phase culture are aseptically packaged in sterilized plastic bags. The final product has a minimum concentration of the active ingredient, with a viability of at least 3×10⁸ colony forming units per mL. The fermented culture of strain CGAPGPBBS-034 is grown under optimized conditions to ensure maximum bacterial viability and retention of plant growth-promoting properties. The liquid formulation stored in sterile bags can be preserved for viable count analysis. Notably, when the liquid formulation is subjected to different temperatures, CGAPGPBBS-034 exhibits a response to temperature variation. Shelf-life studies demonstrate that even after 18 months of storage at both temperatures, the minimum count of bacterial cells remains at 2×10⁸, indicating the sustained viability of the strain.

In another exemplary embodiment, the phytotoxic effects of Bacillus megaterium CGAPGPBBS-034 may assess during a field experiment by monitoring symptoms such as tip injury, wilting, vein clearing, necrosis, and epinasty/hyponasty. Interestingly, no symptoms were observed during or after the application of Bacillus megaterium CGAPGPBBS-034 to all three crops in both seasons. These findings may demonstrate the absence of any harmful effects on the plants, indicating that CGAPGPBBS-034 is an effective plant growth-promoting bacterium suitable for commercial use in both normal and higher pH conditions. Furthermore, it may be utilized in different temperature conditions within Nigeria.

Amplification and 16S rRNA Gene Sequence Analysis: After amplifying the partial 16S rRNA gene, cycle sequencing was performed through MACROGEN in Korea. The resulting amplified product underwent sequencing using the forward sequencing reaction mix. To determine homology, the DNA sequence was searched using the BLAST search engine at the NCBI site (ncbi.nlm.nih.gov) and FASTA (ebi.ac.uk). The FASTA homology search revealed similarity to Bacillus megaterium, and the corresponding strain obtained from NCBI was designated as KY495213.

In another exemplary embodiment of the present disclosure, wherein Bacillus megaterium strain CGAPGPBBS-034 may be was isolated from the alkaline calcareous soil of rhizosphere and rhizoplane from Yola, Nigeria.

In another exemplary embodiment of the present disclosure, wherein Bacillus megaterium strain CGAPGPBBS-034 may be isolated by a serial dilution technique and spread plate method.

In another exemplary embodiment of the present disclosure, Bacillus megaterium strain CGAPGPBBS-034 may capable of phosphate solubilization in higher pH conditions, thereby enabling the availability of phosphorus to plants even in alkaline or high pH soil environments.

In another exemplary embodiment of the present disclosure, Bacillus megaterium strain CGAPGPBBS-034 may has the ability to stimulate plant growth through the production of phytohormones, including indoleacetic acid, thereby regulating plant growth, improving root development, and enhancing overall plant vigor.

In another exemplary embodiment of the present disclosure, wherein the nutrient medium comprises carbon sources selected from the group consisting of sucrose, glucose, and molasses, nitrogen sources selected from the group consisting of ammonium sulfate, urea, and peptone, and phosphate sources selected from the group consisting of calcium phosphate, potassium phosphate, and sodium phosphate, providing optimal conditions for bacterial growth and nutrient assimilation.

In another exemplary embodiment of the present disclosure, wherein the fermentation may be carried out under controlled conditions including temperature, pH, and oxygen levels, to promote optimal bacterial growth and metabolic activity.

In another exemplary embodiment of the present disclosure, wherein the application of the bacterial formulation to soil may be performed using sprinkler irrigation, spray application, drone-based application, soil mixing techniques, seed deepening methods, plant root deepening methods, or any combination thereof.

In another exemplary embodiment of the present disclosure, wherein the bacterial formulation may be mixed with a carrier material selected to provide stability to the bacterial cells in the soil for an extended period, thereby enhancing plant health and promoting long-term effectiveness of the biofertilizer.

Example: 1—Isolation of rhizobacteria: Bacteria were isolated from the alkaline calcareous soil of rhizosphere and rhizoplane from Yola, Nigeria by a serial dilution technique and spread plate method.

Process for isolation and cultivation of Bacteria from Soil Sample:
a) Obtain a sample containing bacteria, either by scraping the soil surface or using a sterile instrument to collect a soil core.
b) Prepare nutrient agar plates by sterilizing them through autoclaving and allowing them to cool to room temperature. These plates will serve the purpose of isolating bacteria from the soil sample.
c) Take a small portion of the soil sample and evenly distribute it across the surface of the nutrient agar plate. Incubate the plates at temperatures ranging from 35-37° C. for a period of 24-48 hours.
d) After the incubation period, bacterial growth should be visible on the nutrient agar plates. Select one or more colonies and streak them onto new nutrient agar plates to obtain pure cultures.
e) Store the pure cultures in 50% glycerol solution for future utilization.

Example: 2—Indole acetic acid test: Indole acetic acid test steps of Bacterial Strain CGAPGPBBS-048:
i. The peptone water was supplemented with tryptophan to prepare indole-producing media.
ii. Select a pure culture of the bacterial strain, and transfer a loopful of the bacterial culture into the corresponding test tubes.
iii. Incubate the inoculated test tubes at 37° C. for 24-48 hours.
iv. add approximately 0.5 mL of Kovacs reagent to detect indole acetic acid to each test tube.
v. Observe the test tubes for the appearance of red color in the Kovacs reagent layer at the top of the broth, and take the optical density (OD) at 530 nm.

Example: 3—Indole acetic acid test in a higher pH medium.
a. Prepare nutrient agar plates and streak the bacterial strain CGAPGPBBS-048 for fresh culture. Incubate the plates at the appropriate temperature for 24-48 hours until visible colonies form.
b. Inoculate a single colony of the bacterial strain CGAPGPBBS-048 into a 5 mL trypticase soya broth and incubate at the appropriate temperature with shaking for 24 hours.
c. Prepare a higher pH medium using Tris-HCI buffer in the peptone water was supplemented with tryptophan to prepare indole-producing media (adjust the pH up to 8.5).
d. The bacterial isolate (0.3 ml inoculum with approximately 108 CFU ml⁻¹) was inoculated into a test tube containing 10 mL of the low pH medium (peptone water with tryptophan). Incubate the test tube at the appropriate temperature with shaking.
e. Incubate the inoculated test tubes at 37° C. for 24-48 hours.
f. Add approximately 0.5 mL of Kovacs reagent to detect indole acetic acid to each test tube.

g. Observe the test tubes for the appearance of red color in the Kovacs reagent layer at the top of the broth, and take the optical density (OD) at 530 nm.

TABLE 1
Indole acetic acid production in a higher pH medium

| | Optical | Indole | | Optical | Indole | |
|---|---|---|---|---|---|---|
| Time | Density | acetic acid | pH of | Density | acetic acid | pH of |
| (days) | (660 nm) | (mg L⁻¹) | medium | (660 nm) | (mg L⁻¹) | medium |
| 1 | 1.5 ± 0.02ef | 120.09 ± 1.40f | 8.31 ± 0.10a | 0.5 ± 0.01e | 15.85 ± 0.18f | 8.67 ± 0.01a |
| 2 | 1.64 ± 0.02d | 126.09 ± 1.47f | 7.8 ± 0.09b | 0.6 ± 0.01d | 19.81 ± 0.23e | 8.6 ± 0.06a |
| 3 | 1.79 ± 0.02bc | 136.1 ± 1.59f | 7.39 ± 0.09b | 0.99 ± 0.01a | 25.76 ± 0.30d | 8.52 ± 0.10ab |
| 4 | 1.94 ± 0.11a | 154 ± 8.48e | 6.42 ± 0.35c | 0.89 ± 0.05b | 28.46 ± 1.57d | 8.31 ± 0.27b |
| 5 | 1.91 ± 0.10ab | 170.42 ± 9.39d | 5.79 ± 0.32d | 0.77 ± 0.04c | 32.52 ± 1.79c | 7.83 ± 0.20c |

Example: 4—Inoculation Effect on cowpea, groundnut, and soybean Seed Emergence
a) Bacterial strain SVRGM/DBT2/03/2020 was grown in 100 ml of Nutrient broth for 48 hours on a rotary shaker. Bacterial cell culture was concentrated by centrifugation and washed with distilled water, and suspended in NaCl solution.
b) Take the surface sterilized legume seeds and place the legume seeds in a petri dish lined with a damp paper towel. Ensure that the seeds are spread out evenly and not touching each other.
c) Add the appropriate amount of inoculant to the seeds in the petri dish. Mix the seeds and inoculant gently to ensure that the seeds are evenly coated.
d) Seal the petri dish with parafilm to maintain moisture and prevent contamination.
e) Place the petri dish in a warm location (30° C.) for 24-48 hours to allow for the inoculant to adhere to the seeds.
f) After the incubation period, plant the inoculated seeds in sterile soil with 3 cm planting depth and spacing for the specific legume species.
g) Water the seeds immediately after planting and ensure that the soil remains moist throughout the germination period.
h) Record the number of seedlings that emerge from the inoculated seeds and compare it to the number of seedlings that emerge from non-inoculated seeds.
i) Calculate the percentage of seedling emergence for both the inoculated and non-inoculated seeds.
j) Analyze the data to determine the effect of inoculation on seedling emergence.
k) Results illustrated that cowpea, groundnut, and soybean seeds inoculated with strain SVRGM/DBT2/03/2020 enhanced seed emergence by 30 to 35% compared to the control.

Example: 5—Effect of PGPR Inoculation on Cowpea, groundnut, and Soybean Yield in Field Trials during the rainy and dry season
i. The trial was conducted in the 2016 rainy season from July to December 2016 and in 2017 dry season from January to June 2017 at two different locations using strain CGAPGPBBS-034 with three crops cowpea, groundnut, and soybean.
ii.The field trials were planted through a commercial seed planter, and foliar bacterial treatment was applied using 2 a handheld sprayer of 250-300 litters per hectare.
iii.All field trials were conducted in the randomized complete block design (RCBD), each crop trials were a size of 10 acres, with bacterial treatment along with two kinds of control (chemical fertilizer control and untreated control), replicated three times. The treatments were designed to evaluate the bacterial ability to enhance crop yield.
iv.Crops were harvested at maturity, seed was collected and cleaned, and yield was measured.
v.Results illustrated that cowpea, groundnut, and soybean yield significantly enhanced as compared to controls (Table 2).

TABLE 2
Result of example 5 Effect of PGPR Inoculation on Cowpea, groundnut,
and Soybean Yield in Field Trials during the Rainy and dry season

| | Location 1 | | | Location 2 | | |
|---|---|---|---|---|---|---|
| Treatments | Cowpea | Groundnut | Soybean | Cowpea | Groundnut | Soybean |

Rainy season

| | | | | | | |
|---|---|---|---|---|---|---|
| CGAPGPBBS- | 1525.57 ± | 1944.55 ± | 1685.44 ± | 1287.17 ± | 1922.11 ± | 1878.69 ± |
| 034 | 70.07a | 85.95a | 85.97a | 56.89a | 78.42a | 81.18a |
| Chemical | 1369.18 ± | 912.93 ± | 1399.53 ± | 1084.70 ± | 1585.98 ± | 1712.80 ± |
| control | 62.89b | 40.35b | 71.38b | 47.94b | 64.71b | 74.01b |
| Untreated | 846.36 ± | 658.45 ± | 1289.06 ± | 955.68 ± | 1383.97 ± | 1178.09 ± |
| control | 38.87c | 29.10c | 65.75b | 42.24c | 56.47c | 50.91c |

Dry season

| | | | | | | |
|---|---|---|---|---|---|---|
| CGAPGPBBS- | 2388.60 ± | 1821.00 ± | 2681.45 ± | 2364.74 ± | 1782.02 ± | 2295.40 ± |
| 034 | 108.49a | 104.48a | 122.60a | 109.19a | 80.11a | 111.62a |
| Chemical | 2163.68 ± | 1593.35 ± | 1947.63 ± | 1964.93 ± | 1547.64 ± | 1929.60 ± |
| control | 98.27b | 91.42b | 89.05b | 90.73b | 69.57b | 93.83b |
| Untreated | 2004.92 ± | 1501.45 ± | 1748.41 ± | 1656.29 ± | 1288.26 ± | 1596.66 ± |
| control | 91.06b | 86.14b | 79.94c | 76.48c | 57.91c | 77.64c |

Columns marked with the same alphabetical letter(s) within comparable means (n = 3) in the same column do not differ significantly using the revised least significant difference (LSD) test at p = 0.05 levels; Mean ± standard deviation

Example: 6—Identification process of Rhizobacterial Strain
a) The bacterial strain CGAPGPBBS-034 was grown on nutrient agar media for 24 hours. Then the bacterial DNA was extracted.
b) The partial 16S rRNA gene was amplified, then subjected to cycle sequencing through MACROGEN, Korea.
c) The amplified product was sequenced using the forward sequencing reaction mix. The DNA sequence was searched for homology using BLAST search engine at NCBI site (ncbi.nlm.nih.gov) and FASTA (ebi.ac.uk).
d) In the FASTA homology search showed similarity towards the Bacillus megaterium, and the strain obtained from NCBI was designated as KY495205.

Example: 7—Commercial Exploitation (viability and phytotoxicity): The Bacillus megaterium strain CGAPGPBBS-034 may demonstrates the ability to solubilize phosphate in alkaline soils and positively influence the germination and yield of diverse crop varieties. Although some rhizospheric microbes may possess advantageous traits for other crops, the presence of specific bacterial populations in the rhizosphere, without modification or supplementation, is primarily may be determined by the availability of substrates, prevailing environmental conditions (such as soil moisture, pH, and organic matter content), and the competition among different microbial communities. These beneficial microbes may establish colonization on plant roots, enhancing nutrient absorption, synthesizing growth hormones, and providing protection against diseases, thereby promoting overall plant growth and health.

We claim:

1. A carrier-based agricultural biofertilizer composition, comprising:
an isolated Bacillus megaterium strain CGAPGPBBS-034 having the deposit accession number KY495205; and
an agriculturally acceptable liquid formulation with a stabilizer to provide stability and support for at least one of the Bacillus species, thereby enhancing the growth and overall health of the plants.

2. The composition as claimed in claim 1, wherein Bacillus megaterium strain CGAPGPBBS-034 was isolated from the alkaline calcareous soil of rhizosphere and rhizoplane from Yola, Nigeria.

3. The composition as claimed in claim 1, wherein Bacillus megaterium strain CGAPGPBBS-034 was isolated by a serial dilution technique and spread plate method.

4. The composition as claimed in claim 1, Bacillus megaterium strain CGAPGPBBS-034 is capable of phosphate solubilization in higher pH conditions, thereby enabling the availability of phosphorus to plants even in alkaline or high pH soil environments.

5. The composition as claimed in claim 1, Bacillus megaterium strain CGAPGPBBS-034 has the ability to stimulate plant growth through the production of phytohormones, including indoleacetic acid, thereby regulating plant growth, improving root development, and enhancing overall plant vigor.

6. A method for enhancing soil using a biofertilizer composition, comprising the steps of:
preparing a nutrient medium containing carbon, nitrogen, and phosphate sources suitable for bacterial growth;
inoculating the nutrient medium with a pure culture of Bacillus megaterium strain CGAPGPBBS-034, having the deposit accession number KY495205, and allowing it to undergo fermentation under controlled conditions;
harvesting the bacterial cells from the fermented broth using appropriate separation techniques;
fermenting the broth at a concentration of 3.0×10\hat{ }9 CFU/mL and zero total dissolved solids (TDS) water, incorporating polyvinyl pyrrolidone (PVP) at a concentration of 0.5%, to obtain a bacterial formulation;
diluting the bacterial formulation to achieve a desired microbial concentration of 3×10\hat{ }8 CFU/mL; and
applying the bacterial formulation to soil by suitable means, ensuring adequate distribution, thereby enhancing the soil.

7. The method as claimed in claim 6, wherein the nutrient medium comprises carbon sources selected from the group consisting of sucrose, glucose, and molasses, nitrogen sources selected from the group consisting of ammonium sulfate, urea, and peptone, and phosphate sources selected from the group consisting of calcium phosphate, potassium phosphate, and sodium phosphate, providing optimal conditions for bacterial growth and nutrient assimilation.

8. The method as claimed in claim 6, wherein the fermentation is carried out under controlled conditions including temperature, pH, and oxygen levels, to promote optimal bacterial growth and metabolic activity.

9. The method as claimed in claim 6, wherein the application of the bacterial formulation to soil is performed using sprinkler irrigation, spray application, drone-based application, soil mixing techniques, seed deepening methods, plant root deepening methods, or any combination thereof.

10. The method as claimed in claim 6, wherein the bacterial formulation is mixed with a carrier material selected to provide stability to the bacterial cells in the soil for an extended period, thereby enhancing plant health and promoting long-term effectiveness of the biofertilizer.
